(19) United States
(12) Patent Application Publication
(10) Pub. No.: US 2007/0031512 A1
(43) Pub. Date: Feb. 8, 2007

(54) Virus-interacting layered phyllosilicates and methods of inactivating viruses

(75) Inventor: John Hughes, University Park, FL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMCOL International Corporation, Arlington Heights, IL (US)

(21) Appl. No.: 11/196,090

(22) Filed: Aug. 3, 2005

(51) Int. Cl. A61K 33/06; A01N 59/06

(52) U.S. Cl. 424/684

(57) ABSTRACT

Layered phyllosilicates are useful for adsorbing and/or binding to and, thereby, inactivating viruses. The layered phyllosilicates can be sprayed into a person's nostrils or contained on a face mask to prevent infection; can be suspended in water for skin contact for virus inactivation; can form a portion of an HVAC filter to prevent virus transfer from room to room, e.g., in a hospital; and can be absorbed in a paper or fabric wipe for inactivating viruses on substrates, such as hospital and operating room furniture and surgical apparatus.

### FIELD

Described herein are virucidal layered phyllosilicates capable of interacting with and thereby inactivating significant percentages of bacteria and a plurality of viruses, particularly HIV and influenza A viruses.

### BACKGROUND

The number of people who were infected with HIV rose to its highest level ever in 2004. The WHO estimated a global total of 39.4 million people living with HIV and that 3.1 million people died of the infection in 2004 (www.unaids.org/wad2004/report.html). Of the world's HIV-infected individuals 50% with teenage girls accounting for 30% of the HIV infected women in some sub-Saharan African countries. Although contraception is available, the HIV epidemic continues to spread highlighting the urgent need for new prevention strategies (Balzarini, J. 2005). Virucides are of interest because they can act quickly and are more direct by binding to the virus coat proteins or viral membranes on contact (Al-Jabri, A. A et al., 2000). A number of HIV virucides are currently under investigation including the physical method of absorbing the virus using mineral clays, a method tried and tested by a number of scientists (Quignon, F. et al. 1997; Clark, K. J., Sarr, A. B., Grant, P. G., Phillips, T. D. & Woode, G. N., 1998; Meschke, J. S. & Sobsey, M. D., 2003). The adsorption effects of bentonite clay in the adsorption of viruses (Sobsey, M. D. and Cromeans, T., 1985; Lipson, S. M. & Stotzky, G., 1985), for example, have been studied extensively in the last few decades due to its use in microbial filtration in the treatment of water.

Further, in the past century we have witnessed three pandemics of influenza, of which the “Spanish flu” of 1918 was the largest pandemic of any infectious disease known to medical science (Oxford, J. S., 2000). The three strains which caused these pandemics belong to group A of the influenza viruses and, unlike the other two groups (B and C), this group infects a vast variety of animals (poultry, swine, horses, humans and other mammals).

Influenza A viruses continue to cause global problems, both economically and medically (Hayden, F. G. & Palese, P., 2000). The recent South East Asian outbreaks of avian influenza in 2003 and 2004 are ideal examples of this.

Much has been done to control and prevent another pandemic from occurring with many anti-influenza products (vaccines and treatments) currently on the market. The most recognized of these is TAMEFLU® (oseltamivir phosphate), a neuraminidase inhibitor, which functions by preventing spread of the virus within the human body.

Scientists have, in the recent years, been looking to develop new drugs following novel strategies of coping with Influenza. With the numbers of such projects on the rise researchers have been focusing on different Influenza target sites in which to develop new vaccines and treatments. Fiers, W. et al. (2004), for example, have reported the efficacy of an M2e vaccine, which targets the less variable M2 transmembrane protein of the influenza virus. Another example is the “OX40 treatment”, which reduces the excessive immune response that accompanies Influenza infections and which can increase the severity of symptoms (Hussell, T. et al. (2004).

Layered phyllosilicates, such as bentonite clay, or montmorillonite clay, are the active virus-interacting minerals described herein for inactivating viruses. Their virus sorption/binding properties, in prior art theory, are due to their negative electrical charge, which attracts positively charged toxins (including bacteria and viruses) and binds them. The virucidal phyllosilicates described herein, however, bind both positively charged and negatively charged virus molecules. It is theorized that sorption and/or binding of the virus to the layered phyllosilicates described herein is achieved by one or more mechanisms selected from the group consisting of adsorption; ionic complexing; electrostatic complexing; chelation; hydrogen bonding; ion-dipole; dipole/dipole; Van Der Waals forces; and any combination thereof. Such ionic bonding, e.g., via one or more cations or negative charge sites of the phyllosilicate sharing electrons with one or two atoms of one or two polar ends of a virus molecule, on an inner surface of phyllosilicate platelet surfaces, provides inactivation of a surprisingly high percentage of the virus molecules.

### SUMMARY

It has been found that layered phyllosilicates are useful for adsorbing and/or binding to and, thereby, inactivating viruses, particularly both the human immunodeficiency virus (HIV) and influenza A virus. The ability of a layered phyllosilicate to interact with and inactivate two very different acting viruses is most unexpected.

The layered phyllosilicate material useful for virus interaction, as described herein, includes the following clay minerals: montmorillonite, particularly sodium montmorillonite, magnesium montmorillonite and/or calcium montmorillonite; nontronite; beidellite; laponite; yakhontovite; zincsilite; volkonskoite; hectorite; saponite; ferrosaponite; sauconite; swinefordite; pimelite; sobockite; stevensite; svinfordite; vermiculite; synthetic clays; mixed layered illite/smectite minerals, such as rectorite, tarosovite, and ledikite; admixtures of illites with the clay minerals named above, and the magnesium aluminum silicates. Any one or any mixture of two or more of the above clay minerals is capable of adsorbing, and/or ionically bonding with, any virus, or combination of viruses, thereby inactivating the virus(es).

One preferred layered phyllosilicate is a smectite clay having at least 80%, preferably at least 95% interlayer, exchangeable homoionic cations, preferably sodium ions, based on the total of number of interlayer, exchangeable cations. Other particularly-effective phyllosilicates that are effective in interacting with and inactivating significant percentages of a host of viruses, particularly HIV and influenza A viruses, include protonated onium ion-exchanged layered phyllosilicates (protonated organoclays); smectite clays having a particle size less than 74 μm, preferably less than 50 μm, more preferably less than 20 μm; and exfoliated smectite clays, including individual clay platelets and tactoids of 5 or less platelet layers.

In accordance with one embodiment for using the virucidal layered phyllosilicates-described herein, the phyllosilicate particles are sprayed onto an absorbent mask as an air purification device, or included in a hand wipe material (hand sanitizers) for cleaning virus-contaminated surfaces, thereby adsorbing and inactivating the viruses, thereby preventing viruses from being breathed into the nose and mouth of a person or for adsorbing and thereby inactivating viruses from the hands, e.g., before handling a baby; or on gloves to inactivate viruses.

In other embodiments, the virucidal layered phyllosilicates can be suspended in lotions or skin creams that are applied to skin, particularly hands and face, or internally within the vagina, for interacting with and thereby inactivating the transfer of viruses from one person to another, or to prevent a person from transferring the virus from external skin to internal cells.

In still another embodiment, the virucidal layered phyllosilicates can be ingested for internal interaction and inactivation of viruses within the gastrointestinal tract that have been or are about to be ingested. When wastes are expelled, viruses are retained on the clay and prevented from causing secondary infections.

In another embodiment, the virucidal layered phyllosilicates can be vaginally inserted for interaction and inactivation of HIV or other sexually-transmitted viruses, in the same manner as a spermicidal foam or body heat-dissolving spermicidal cartridge.

In still another embodiment, the virucidal layered phyllosilicates can be held in a vessel for filtering contact with blood, e.g., a secondary dialysis filter, or for filtering viruses from water in a virus-removing water purification step.

In another embodiment, the virucidal layered phyllosilicates can be used as, or form a portion of, a HVAC filtration media to prevent virus-contaminated air from passing between rooms; for example, between rooms in a hospital.

In another embodiment, the virucidal layered phyllosilicates are used as a nasal lubricant by spraying a suspension of the virucidal phyllosilicate in a carrier (water and/or organic solvent) into the nasal passages to coat nasal cells. In this manner, viruses entering the nose will interact with the phyllosilicate and thereby will be inactivated to prevent infection.

In still another embodiment, a condom is coated with a suspension of the virucidal layered phyllosilicates; in a cosmetically acceptable carrier, e.g., water and/or solvent. In the event of condom failure, the virucidal phyllosilicate interacts with and inactivates viruses before a sexual partner is infected.

In another embodiment, a suspension of the virucidal layered phyllosilicate in a cosmetically acceptable carrier is packaged in a portable container, e.g., a tube or bottle, for use on the hands to periodically inactivate viruses held on a person's skin.

In another embodiment, the virucidal layered phyllosilicates can be dispensed throughout a virus-contaminated body of water, such as a pond or lake, to inactivate viruses therein.

The virucidal layered phyllosilicates described herein interact with viruses, adsorb and/or bind them ionically to the virucidal layered phyllosilicates, thereby preventing the viruses from migrating to and penetrating cell membranes, thereby preventing the viruses from reproducing and rupturing the cells and releasing more of the virus.

Whenever used in this specification, the terms set forth shall have the following meanings:

Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

“Phyllosilicate” or “Virucidal Clay”: shall mean clay minerals, e.g., montmorillonite, particularly sodium montmorillonite, magnesium montmorillonite and/or calcium montmorillonite; nontronite; beidellite; laponite; yakhontovite; zincsilite; volkonskoite; hectorite; saponite; ferrosaponite; sauconite; swinefordite; pimelite; sobockite; stevensite; svinfordite; vermiculite; synthetic clays; mixed layered illite/smectite minerals, such as rectorite, tarosovite, and ledikite; admixtures of illites with the clay minerals named above, and the magnesium aluminum silicates.

“Homoionic Phyllosilicate” shall mean a layered Phyllosilicate material that has been purified by ion-exchange, for example, as described in this assignee's U.S. Pat. No. 6,050,509, to contain at least 90% of a single element, in relation to all interlayer exchangeable cations, from periodic table groups 1a, 2a, 3b, 4b, 5b, 6b, 7b, 8, 1b, 2b, 3a, tin and lead; or a protonated onium ion compound, as the interlayer exchangeable cations.

“Platelets” shall mean individual layers of a Phyllosilicate.

“Intercalate” or “Intercalated” shall mean a phyllosilicate material that includes an onium ion spacing agent, preferably a protonated onium ion spacing agent, disposed between adjacent platelets of the layered Phyllosilicate material to increase the interlayer spacing between the adjacent platelets by at least 3 Å, preferably at least 5 Å, to an interlayer spacing, for example, of at least about 8 Å, preferably at least about 10 Å.

“Intercalation” shall mean a process for forming an Intercalate.

“Onium Ion Intercalant” or Onium Ion Spacing Agent” or “Onium Ion Compound” shall mean an organic compound, preferably a protonated organic compound, that includes at least one positively charged atom selected from the group consisting of a nitrogen atom, a phosphorous atom, a sulfur atom or an oxygen atom, preferably a quaternary ammonium compound, and when dissolved in water and/or an organic solvent, an anion dissociates from the onium ion spacing agent leaving an onium cation that can ion-exchange with a silicate platelet exchangeable cation of the Phyllosilicate, e.g., Na⁺, Ca⁺², Li⁺, Mg⁺², Al⁺³, or K⁺.

“Intercalating Carrier” shall mean a carrier comprising water and/or an organic liquid to form an Intercalating Composition capable of achieving Intercalation of an onium ion spacing agent which ion-exchanges with exchangeable interlayer cations of the layered Phyllosilicate.

“Intercalating Composition” shall mean a composition comprising one or more onium ion spacing agents, an Intercalating Carrier for the onium ion spacing agent, and a layered Phyllosilicate.

“Exfoliate” or “Exfoliated” shall mean individual platelets of an Intercalated layered Phyllosilicate so that adjacent platelets of the Intercalated layered Phyllosilicate can be dispersed individually throughout a carrier material, such as water, a polymer, an alcohol or glycol, or any other organic liquid, together with tactoids of 2-20 layers of non-exfoliated platelets.

Exfoliation” shall mean a process for forming an Exfoliate from an Intercalate.

### Clay Purification and Ion-Exchange

A preferred layered phyllosilicate useful for interaction with an inactivation of viruses is a smectite clay that has been purified and ion-exchanged in accordance with this assignee's U.S. Pat. No. 6,050,509, hereby incorporated by reference. The ion-exchange process can be used to provide a homoionic layered phyllosilicate or can be used to provide the phyllosilicate with mixed cations from the periodic table groups 1a, 1b, 2a, 2b, 3a, 3b, 4b, 5b, 6b, 7b, 8, tin, hydrogen, lead, and/or protonated onium ions, within any percentage of the phyllosilicate exchangeable cations (1-99% of the exchangeable cations). According to U.S. Pat. No. 6,050,509 the smectite clay slurry is pumped to a series of ion exchange columns where any undesirable cation is exchanged with a desirable cation. In this manner, the crude montmorillonite clay can be exchanged to produce a purified montmorillonite with a single (homoionic) desirable cation or with a mixture of cations. In this manner, by using the appropriate ion exchange column, any element can be exchanged for the interlayer cations of a phyllosilicate for virus inactivation, including hydrogen and/or one or more elements from the following groups of the periodic table: group 1a (e.g., lithium, sodium, potassium) group 2a (e.g., magnesium, calcium, barium) group 3b (e.g., lanthanium), group 4b (e.g., titanium) group 5b (e.g., vanadium), group 6b (e.g., chromium), group 7b (e.g., manganese) group 8 (e.g., iron, cobalt, nickel, platinum), group 1b (e.g., copper, gold, silver), group 2b (e.g., zinc, cadmium) group 3a (e.g., boron, aluminum) and selected members of group 4a (e.g., tin and lead). In this manner, one could exchange a metal or metal cation with known, good antimicrobial or antiviral properties on the surface of the montmorillonite clay, or any layered phyllosilicate material, to produce a material with superior antimicrobial and antiviral properties. Homoionic hydrogen ion-exchanged layered phyllosilicates are formed as follows: (1) A slurry of 1% by weight of sodium montmorillonite clay in de-ionized water was prepared; (2) The 1% by weight sodium montmorillonite slurry was pumped through an ion-exchange column filled with hydrogen ion-exchange beads. The hydrogen ion-exchange beads were formed by contacting ion-exchange beads with an excess of 2N HCl; and (3) The hydrogen ion-exchanged slurry was diluted to 0.1% by weight for testing.

In accordance with this embodiment of the virucidal layered phyllosilicate, the crude layered phyllosilicate deposits initially include one or more of the following non-smectite impurities: (SiO₂), feldspar (KAISi₃ O₈), opal-CT (SiO₂); gypsum (CaSO₄.2H₂O); albite (NaAlSi₃O₈); anorthite (CaAl₁₂Si₂O₈); orthoclase (KAlSi₃O₈); apatite (Ca₅(PO₄)₃(F,Cl,OH)); halite (NaCl); calcite (CaCO₃); dolomite (CaMg(CO₃)₂; sodium carbonate (Na₂CO₃); siderite (FeCO₃) biotite (K(Mg,Fe)₃(AlSi₃O₁₀) (OH)₂) muscovite (KAl₂(AlSi₃O₁₀) (OH)₂); chlorite ((Mg,Fe)₆(Si,Al)₄O₁₀ (OH)₈); stilbite (NaCa₂Al₅Si₁₃O₃₆.14H₂O); pyrite (FeS₂); kaolinite (Al₂Si₂O₅.(OH)₄); and hematite (Fe₂O₃)

In order to remove at least 90% by weight of the above impurities, preferably at least 99% of the impurities, preferably, the layered phyllosilicate is dispersed in water, preferably at a concentration of about 10% to about 15% by weight, based on the total weight of phyllosilicate and water. The preferred layered phyllosilicate is a smectite clay, such as a montmorillonite clay, that is predominantly (greater than about 50% by weight) sodium or calcium montmorillonite clay so that the concentration of clay dispersed in water can be as high as about 15% by weight. If, for example, a sodium montmorillonite clay is dispersed in water, the higher swelling capacity of sodium montmorillonite in water will result in a viscosity that is too high for handling at a concentration of about 6-10% by weight. Accordingly, in order to achieve the most efficient purification of the smectite clay, it is preferred that the clay dispersed in water is a montmorillonite clay having predominantly (at least 50% by number) multivalent cations, i.e., Ca⁺² in the interlayer space, such as calcium montmorillonite clay. If the clay is not predominantly a multivalent clay, such as calcium montmorillonite, it can be ion-exchanged sufficiently to provide predominantly multivalent ions in the interlayer spaces between montmorillonite clay platelets.

The clay slurry is then directed into a series of cascaded hydrocyclones of decreasing size, each hydrocyclone capable of removing impurities of at least a particular size, particularly the impurities having a size greater than about 74 microns. The resulting clay, separated from the impurities, has a particle size such that at least about 90% by volume of the clay particles have a size below about 74 microns, preferably below about 50 microns, more preferably below about 20 microns. The clay slurry is then directed upwardly through a cation exchange column that removes multivalent interlayer cations from the montmorillonite clay (e.g., divalent and/or trivalent cations) and substitutes monovalent cations such as sodium, lithium and/or hydrogen for the multivalent cations within the interlayer spaces between platelets of the montmorillonite clay.

After essentially complete ion exchange, such that the clay has at least 90%, preferably at least 95%, more preferably at least 99%, by number, monovalent cations in the interlayer spaces, the clay preferably is then directed into a high speed centrifuge where the clay is subjected to centrifugal force equal to, for example, at least about 2,000 G (forces of gravity) up to about 4,000 G, preferably about 2,500 G to about 3,500 G, capable of removing clay particle sizes between about 5 microns and about 74 microns, such that the remaining montmorillonite clay particles, having less than about 50 by weight crystalline and amorphous non-smectite clay impurities, preferably less than about 5% by weight impurities therein, have a particle size of about 10 microns or less, preferably about 8 microns or less, and have an average particle size less than about 3 microns, preferably less than about 2 microns.

In accordance with an important feature of this embodiment, for effective removal of the impurities that have a size less than about 10 microns in diameter, the clay should first be conditioned or treated for removal of all multivalent, e.g., divalent and trivalent, interlayer cations by substitution of the multivalent cations with one or more monovalent cations, such as sodium ions, or protonated onium ions, in order to provide effective removal of the smallest impurities, for example, in a high speed (2,000 G) centrifuge. In accordance with another important feature of this embodiment, it has been found that conveying the clay slurry through the hydrocyclones prior to monovalent, e.g., sodium ion-exchange provides for a much more efficient process since the material fed to the hydrocyclones can be fed at a higher solids content without an undue increase in the viscosity of the material fed to the hydrocyclones. Accordingly, ion-exchange is accomplished after the clay slurry is passed through the hydrocyclones and before sending the partially purified clay slurry to a centrifuge for removal of the smallest impurities removed from the product.

The product from primary and secondary one inch hydrocyclones are fed by gravity to an ion-exchange feed tank where the clay/water slurry, including impurities, are maintained at a clay concentration of about 1-7% by weight, preferably about 3-7% by weight, based on the total weight of material in the ion-exchange feed tank. The clay slurry from the ion-exchange feed tank is pumped to a series of ion-exchange columns where the interlayer clay cations are exchanged with cations from periodic table groups 1a, 1b, 2a, 2b, 3a, 3b, 4b, 5b, 6b, 7b, 8, tin or lead, preferably sodium. Ion-exchange is achieved, for example, by contact with an ion-exchange resin, preferably PUROLITE C-100, obtained from The PUROLITE Company, a polystyrene cross linked with divinyl benzene, in spherical bead form, in the sodium ionic form, having an 8% by weight divinyl benzene content.

The product from a secondary one inch hydrocyclone includes at least about 90% by number particles having a size less than about 50 microns, preferably less than about 20 microns, more preferably less than about 10 microns, a mean particle size less than about 10 microns, and a median particle size less than about 5 microns.

### Exfoliated Clay to Form Clay Platelets and/or Tactoids

To form the intercalated and exfoliated layered phyllosilicates described herein, the phyllosilicate material, e.g., bentonite, should be swelled or intercalated, in the preferred embodiment, by sorption of an onium ion spacing agent.

While the compositions and methods described herein are described by way of the preferred embodiment via expanding the interlaminar spacing between adjacent platelets of a layered phyllosilicate material by intercalating onium ions between the silicate platelets, the interlaminar spacing also can be achieved by intercalating a silane coupling agent, or by an acidification technique, by substitution, with hydrogen (ion-exchanging the interlayer cations with hydrogen by use of an acid or ion-exchange resin) as disclosed in the Deguchi U.S. Pat. No. 5,102,948, and in the Lan, et al. U.S. Pat. No. 5,853,886, both patents hereby incorporated by reference. In this clay exfoliation embodiment, the extremely small size of the individual platelets and clay tactoids should permit interaction, with and inactivation of all viruses, including neoviruses, poliovi uses type 2, euteroviruses, bovine rotavirus, and bovine corona viruses.

Sorption of the onium ion spacing agent should be sufficient to achieve expansion of the interlayer spacing of adjacent platelets of the layered phyllosilicate material (when measured dry) by at least about 3 Å, preferably at least about 5 Å.

The onium ion spacing agent is introduced into the layered phyllosilicate galleries in the form of a solid or liquid composition (neat or aqueous, with or without an organic solvent, e.g., an aliphatic hydrocarbon, such as heptane to, if necessary, aid to dissolve the onium ion compound) having an onium ion spacing agent concentration sufficient to provide a concentration of about 5% to about 10% by weight phyllosilicate (90-95% water) and the onium ion compound is dissolved in the phyllosilicate slurry water, preferably at a molar ratio of onium ions to exchangeable interlayer cations of at least about 0.25:1, more preferably at least about 0.5:1, most preferably at least about 1:1. The onium ion-intercalated layered phyllosilicate then is separated from the water easily, since the phyllosilicate is now hydrophobic, and dried in an oven to less than about 15% water, preferably bone dry, before interaction with the virus. The onium ion spacing agent compound can be added as a solid with the addition to the layered phyllosilicate material/onium ion compound blend of preferably at least about 20% water, more preferably at least about 30% water or more, based on the dry weight of layered material. Preferably about 30% to about 50% water, more preferably about 30% to about 40% water, based on the dry weight of the layered material, is included in the onium ion intercalating composition, so that less water is sorbed by the intercalate, thereby necessitating less drying energy after onium ion intercalation.

The onium ion spacing agent cations intercalated via ion-exchange into the interlayer spaces between adjacent layered material platelets are primary, secondary, tertiary or quaternary onium ions having the following preferred structure: 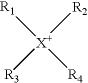
wherein X═N, P, S, or O; and
wherein R₁, R₂, R₃ and R₄ are H or organic moieties, such as linear or branched alkyl, aryl or aralkyl moieties having 1 to about 24 carbon atoms.

The more preferred protonated C₆+ onium ions are preferably quaternary ammonium ions having Formula 1, as follows: 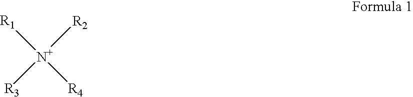
wherein R₁ is a long chain alkyl moiety ranging from C₆ to C₂₄, straight or branched chain, including mixtures of long chain moieties, i.e., C₆, C₈, C₁₀, C₁₂, C₁₄, C₁₆, C₁₈, C₂₀, C₂₂ and C₂₄, alone or in any combination; and R₂, R₃ and R₄ are moieties, same or different, selected from the group consisting of H, alkyl, benzyl, substituted benzyl, e.g., straight or branched chain alkyl-substituted and halogen-substituted; ethoxylated or propoxylated alkyl; ethoxylated or propoxylated benzyl, e.g., 1-10 moles of ethoxylation or 1-10 moles of propoxylation. Preferred protonated onium ions include protonated octadecylamine, protonated hexyl amine; protonated octyl amine; protonated tallow amine; protonated tallow diamine; protonated tallow triamine; protonated tallow tetraamine; protonated hydrogenated tallow amine; protonated hydrogenated tallow diamine; protonated hydrogenated tallow triamine; protonated hydrogenated tallow tetraamine; protonated octadecyl amine; and mixtures thereof.
R¹—X⁺R—Y⁺
where X⁺ and Y⁺, same or different, are ammonium, sulfonium, phosphonium, or oxonium radicals such as ⁺NH₃, ⁺NH₂—, ⁺N(CH₃)₃, ⁺N(CH₃)₂—, ⁺N(CH₃)₂(CH₂CH₃), ⁺N(CH₃)(CH₂CH₃)—, ⁺S(CH₃)₃, ⁺S(CH₃)₂—, ⁺P(CH₃)₃, ⁺P(CH₃)₂—, ⁺NH₄, ⁺NH₃—, and the like; R is an organic spacing, backbone radical, straight or branched, preferably having from 2 to 24, more preferably 3 to 10 carbon atoms, in a backbone organic spacing molecule covalently bonded at its ends to charged N⁺, P⁺, S⁺ and/or O⁺ cations and R¹ can be hydrogen, or an alkyl radical of 1 to 22 carbon atoms, linear or branched, preferably having at least 6 carbon atoms. Examples of R include substituted or unsubstituted alkylene, cycloalkenylene, cycloalkylene, arylene, alkylarylene, either unsubstituted or substituted with amino, alkylamino, dialkylamino, nitro, azido, alkenyl, alkoxy, cycloalkyl, cycloalkenyl, alkanoyl, alkylthio, alkyl, aryloxy, arylalkylamino, alkylamino, arylamino, dialkylamino, diarylamino, aryl, alkylsufinyl, aryloxy, alkylsulfinyl, alkylsulfonyl, arylthio, arylsulfinyl, alkoxycarbonyl, arylsulfonyl, or alkylsilane. Examples of R1 include non-existent; H; alkyl having 1 to 22 carbon atoms, straight chain or branched; cycloalkenyl; cycloalkyl; aryl; alkylaryl, either unsubstituted or substituted or substituted with amino, alkylamino, dialkylamino, nitro, azido, alkenyl, alkoxy, cycloalkyl, cycloalkenyl, alkanoyl, alkylthio, alkyl, aryloxy, arylalkylamino, alkylamino, arylamino, dialkylamino, diarylamino, aryl, alkylsufinyl, aryloxy, alkylsulfinyl, alkylsulfonyl, arylthio, arylsulfinyl, alkoxycarbonyl, arylsulfonyl, or alkylsilane. Illustrative of useful R groups are alkylenes, such as methylene, ethylene, octylene, nonylene, tert-butylene, neopentylene, isopropylene, sec-butylene, dodecylene and the like; alkenylenes such as 1-propenylene, 1-butenylene, 1-pentenylene, 1-hexenylene, 1-heptenylene, 1-octenylene and the like; cycloalkenylenes such as cyclohexenylene, cyclopentenylene and the like; alkanoylalkylenes such as butanoyl octadecylene, pentanoyl nonadecylene, octanoyl pentadecylene, ethanoyl undecylene, propanoyl hexadecylene and the like; alkylaminoalkylenes, such as methylamino octadecylene, ethylamino pentadecylene, butylamino nonadecylene and the like; dialkylaminoalkylene, such as dimethylamino octadecylene, methylethylamino nonadecylene and the like; arylaminoalkylenes such as phenylamino octadecylene, p-methylphenylamino nonadecylene and the like; diarylaminoalkylenes, such as diphenylamino pentadecylene, p-nitrophenyl-p-α-methylphenylamino octadecylene and the like; alkylarylaminoalkylenes, such as 2-phenyl-4-methylamino pentadecylene and the like; alkylsulfinylenes, alkylsulfonylenes, alkylthio, arylthio, arylsulfinylenes, and arylsulfonylenes such as butylthio octadecylene, neopentylthio pentadecylene, methylsulfinyl nonadecylene, benzylsulfinyl pentadecylene, phenylsulfinyl octadecylene, propylthiooctadecylene, octylthio pentadecylene, nonylsulfonyl nonadecylene, octylsulfonyl hexadecylene, methylthio nonadecylene, isopropylthio octadecylene, phenylsulfonyl pentadecylene, methylsulfonyl nonadecylene, nonylthio pentadecylene, phenylthio octadecylene, ethyltio nonadecylene, benzylthio undecylene, phenethylthio pentadecylene, sec-butylthio octadecylene, naphthylthio undecylene and the like; alkoxycarbonylalkylenes such as methoxycarbonylene, ethoxycarbonylene, butoxycarbonylene and the like; cycloalkylenes such as cyclohexylene, cyclopentylene, cyclo-octylene, cycloheptylene and the like; alkoxyalkylenes such as methoxy-methylene, ethoxymethylene, butoxymethylene, propoxyethylene, pentoxybutylene and the like; aryloxyalkylenes and aryloxyarylenes such as phenoxyphenylene, phenoxymethylene and the like; aryloryalkylenes such as phenoxydecylene, phenoxyoctylene and the like; arylalkylenes such as benzylene, phenthylene, 8-phenyloctylene, 10-phenyldecylene and the like; alkylarylenes such as 3-decylphenylene, 4-octylphenylene, 4-nonylphenylene and the like; and polypropylene glycol and polyethylene glycol substituents such as ethylene, propylene, butylene, phenylene, benzylene, tolylene, p-styrylene, p-phenylmethylene, octylene, dodecylene, octadecylene, methoxy-ethylene, moieties of the formula —C₃H₆COO—, —C₅H₁₀COO—, —C₇H₁₀COO—, —C₇H₁₄COO—, —C₉H₁₈COO—, —C₁₁H₂₂COO—, —C₁₃H₂₆COO—, —C₁₅H₃₀COO—, and —C₁₇H₃₄COO— and —C═C(CH₃)COOCH₂CH₂—, and the like. Such tetra-, tri-, and di-ammonium, -sulfonium, -phosphonium, -oxonium; ammonium/sulfonium; ammonium/phosphonium; ammonium/oxonium; phosphonium/oxonium; sulfonium/oxonium; and sulfonium/phosphonium radicals are well known in the art and can be derived from the corresponding amines, phosphines, alcohols or ethers, and sulfides.

Other useful spacing agent compounds are multi-onium ion compounds that include at least two primary, secondary, tertiary or quaternary ammonium, phosphonium, sulfonium, and/or oxonium ions having Formula 2, as follows: 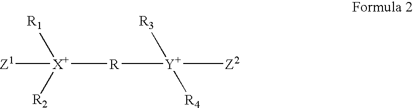
wherein R is an alkylene, aralkylene or substituted alkylene charged atom spacing moiety, preferably ranging from C₃ to C₂₄, more preferably about C₃ to C₆ for relatively high charge density (150 milliequivalents/100 grams C.E.C. to 70 milliequivalents/100 grams C.E.C.) layered materials; and preferably from C₆ to C₁₂ for medium to low charge density (70 milliequivalents/100 grams C.E.C. to 30 milliequivalents/100 grams C.E.C.) layered materials. R can be straight or branched chain, including mixtures of such moieties, i.e., C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃ and C₂₄, alone or in any combination; and R₁, R₂, R₃ and R₄ are moieties, same or different, selected from the group consisting of hydrogen, alkyl, aralkyl, benzyl, substituted benzyl, e.g., straight or branched chain alkyl-substituted and halogen-substituted; ethoxylated or propoxylated alkyl; ethoxylated or propoxylated benzyl, e.g., 1-10 moles of ethoxylation or 1-10 moles of propoxylation. Z¹ and Z², same or different, may be non-existent, or may be any of the moieties described for R₁, R₂, R₃ or R₄. Also, one or both of Z¹ and Z² may include one or more positively charged atoms or onium ion molecules.

Any swellable layered phyllosilicate material that sufficiently sorbs the onium ion spacing agent to increase the interlayer spacing between adjacent phyllosilicate platelets by at least about 3 Å, preferably at least about 5 Å, can be used in the practice of this invention. Useful swellable layered materials include phyllosilicates, such as smectite clay minerals, e.g., montmorillonite, particularly sodium montmorillonite, magnesium montmorillonite, and/or calcium montmorillonite; nontronite; beidellite; laponite; yakhontovite; zincsilite; volkonskoite; hectorite; saponite; ferrosaponite; sauconite; swinefordite; pimelite; sobockite; stevensite; svinfordite; vermiculite; synthetic clays; mixed layered illite/smectite minerals, such as rectorite, tarosovite, and ledikite; admixtures of illites with the clay minerals named above, and the magnesium aluminum silicates.

Preferred swellable layered materials are phyllosilicates of the 2:1 type having a negative charge on the layers ranging from about 0.15 to about 0.9 charges per formula unit and a commensurate number of exchangeable metal cations in the interlayer spaces. Most preferred layered materials are smectite clay minerals such as montmorillonite, nontronite, beidellite, volkonskoite, hectorite, saponite, sauconite, sobockite, stevensite, and svinfordite.

As used herein the “interlayer spacing” refers to the distance between the internal faces of the adjacent phyllosilicate layers as they are assembled in the layered material before any delamination (exfoliation) takes place. The preferred clay materials generally include interlayer cations such as Na⁺, Ca⁺², K⁺, Mg⁺, Al⁺³⁺, NH₄ and the like, including mixtures thereof, and can be ion-exchanged to include other cations such as the elements from period table group 1a, 1b, 2a, 2b, 3a, 3b, 4b, 5b, 6b, 7b, 8, tin and lead.

The onium ions, may be introduced into (sorbed within) the interlayer spaces of the layered phyllosilicate in a number of ways. In a preferred method of intercalating the onium ions between adjacent platelets of the layered material, the phyllosilicate material is slurried in water, e.g., at 5-20% by weight layered phyllosilicate material and 80-95% by weight water, and the onium ion compound is dissolved in the water in which the phyllosilicate material is slurried. If necessary, the onium ion compound can be dissolved first in an organic solvent, e.g., propanol. The phyllosilicate material then is separated from the slurry water and dried suspending the individual silicate platelets and tactoids in a liquid carrier.

To achieve sufficient intercalation of the onium ions between adjacent platelets of the layered phyllosilicate, the phyllosilicate/onium ion intercalating composition preferably contains a molar ratio of onium-ions to layered phyllosilicate of at least 0.25:1, more preferably at least 0.5:1 for the onium ions to exchange interlayer cations with the smectite clay, most preferably 1:1, based on the dry weight of the phyllosilicate, so that the resulting onium ion-intercalated phyllosilicate has interior platelet surfaces that are sufficiently hydrophobic and sufficiently spaced for exfoliation and suspension of the individual platelets and tactoids in a liquid carrier. The onium ion carrier (preferably water, with or without an organic solvent) can be added by first solubilizing or dispersing the onium ion compound in the carrier; or a dry onium ion compound and relatively dry layered phyllosilicate (preferably containing at least about 4% by weight water) can be blended and the intercalating carrier added to the blend, or to the phyllosilicate prior to adding the dry onium ion. When intercalating the phyllosilicate with onium ions in slurry form, the amount of water can vary substantially, e.g., from about 4% by weight, preferably from a minimum of at least about 30% by weight water, with no upper limit to the amount of water in the intercalating composition (the phyllosilicate intercalate is easily separated from the intercalating composition due to its hydrophobicity after onium ion treatment).

Alternatively, the onium ion intercalating carrier, e.g., water, with or without an organic solvent, can be added directly to the phyllosilicate prior to adding the onium ion compound, either dry or in solution. Sorption of the onium ion compound molecules may be performed by exposing the phyllosilicate to a dry or liquid onium ion compound in the onium ion intercalating composition containing at least about 2% by weight, preferably at least about 5% by weight onium ion compound, more preferably at least about 10% onium ion compound, based on the dry weight of the layered phyllosilicate material.

In accordance with an emulsion method of intercalating the onium ions between the platelets of the layered phyllosilicate material, the phyllosilicate, preferably containing at least about 4% by weight water, more preferably about 10% to about 15% by weight water, is blended with water and/or organic solvent solution of an onium ion spacing agent compound in a ratio sufficient to provide at least about 5% by weight, preferably at least about 10% by weight onium ion compound, based on the dry weight of the layered phyllosilicate material.

The onium ion spacing agents have an affinity for the phyllosilicate so that they are sorbed between, and are ion-exchanged with the cations, on the inner surfaces of the silicates platelets, in the interlayer spaces.

### PROTONATED ONIUM ION INTERCALATION EXAMPLES

### Example 1

Example 1 demonstrates the ion exchange process of smectite clay from a Ca form or Na/Ca mixed forms to Na-rich smectite clay.

Raw smectite clay was dispersed into water to make a 3 wt % clay slurry. This clay has a Na content of 0.20 wt % and Ca content of 2.10 wt %. The elemental analysis was measured by an X-ray fluorescence method. The mixture was mixed thoroughly with a mechanical mixer. The pH value of the starting clay slurry is 7-8. An ion exchange resin, such as Amberlite 200C Na, is available from Rohm & Hass packed in a glass column with a 2-in diameter and a 20-in length. A liquid pump was used to pump the clay slurry through the column at 20 ml/min. Elemental analysis of the finished clay, dried from the slurry, indicated that the Na content is 3.45 wt % and Ca content is 0.17 wt %. The ion exchanged clay is called E1-Na-Clay. This clay had a basal spacing of 13 Å.

### Example 2

Example 2 demonstrates the formation of protonated Octadecyl ammonium-treated smectite clay with Octadecyl ammonium acetate from the ion exchanged Na-smectite clay (E1-Na-clay) of Example 1.

100-g of sodium smectite clay E1-Na-clay was dispersed into 3000 ml water through a mechanical mixer. T-his clay slurry was heated to 80° C. 41.5 g of Octadecyl ammonium acetate from KAO Chemicals was added into the clay slurry. The clay showed excellent flocculation after the addition of the Octadecyl ammonium acetate. The pH of the clay reaction slurry was about 4. The clay was filtered with regular quantitative filter paper with the assistance of a mechanical vacuum pump. Then, the clay was dried in an oven over night at 80° C. and ground to pass through a 300-mesh screen as a fine powder. This modified clay was called E2-ODA-Clay.

### Example 3

Example 3 demonstrates the formation of protonated Octadecyl ammonium-treated smectite clay with a solution of Octadecyl ammonium ions in dilute HCl. (E3-ODA-Clay). This sample was measured by powder X-ray diffraction to determine the clay basal spacing after ion exchange. The result is listed in Table-1.

100-g of sodium smectite E1-Na-clay was dispersed into 3000 ml water through a mechanical mixer. This clay slurry was heated to 80° C. 33.8 g of Octadecyl amine was added into 1000 ml of 70° C. water and then mixed with 17.1 g of 10.5 N HCl. The Octadecyl amine-HCl solution was added into the clay slurry followed by mixing. The reaction slurry had a pH of 4. The clay showed excellent flocculation after the addition of the Octadecyl amine-HCl solution. The clay was filtered with regular quantitative filter paper with the assistance of a mechanical vacuum pump. Then, the clay was dried in an oven over night at 80° C. and ground to pass through a 300-mesh screen as a fine powder. This modified clay was called E3-ODA-Clay. This sample was measured by powder X-ray diffraction to determine the clay basal spacing after ion exchange. The result is listed in Table-1.

### Viruses and Viral Taxonomy

Viruses constitute a large and heterogeneous group, and they are classified in hierarchical taxonomic categories based on many different characteristics, e.g., morphology, antigenic properties, physiochemical and physical properties, proteins, lipids, carbohydrates, molecular properties, organization and replication, and biological properties. Whether the RNA or DNA is single or double stranded, the organization of the genome and the presence of particular genes comprise important aspects of the current taxonomy of viruses. All of the former are used to place a virus into a particular order or family. The classification is based upon macromolecules produced (structural proteins and enzymes), antigenic properties and biological properties (e.g., accumulation of virions in cells, infectivity, hemagglutination).

Viral classification is dynamic in that new viruses are continuously being discovered and more information is accumulating about viruses already known. The classification and nomenclature of the latest known viruses appear in reports of the International Committee on the Taxonomy of Viruses (ICTV), 7th edition (van Regenmortel et al., editors. Seventh ICTV report. San Diego: Academic Press; 2000.) The basic viral hierarchical classification scheme is: Order, Family, Subfamily, Genus, Species, Strain, and Type as set out below.

Virus orders represent groupings of families of viruses that share common characteristics and are distinct from other orders and families. Virus orders are designated by names with the suffix-virales. Virus families are designated by names with the suffix-viridae. Virus families represent groupings of genera of viruses that share common characteristics and are distinct from the member viruses of other families. Viruses are placed in families on the basis of many features. A basic characteristic is nucleic acid type (DNA or RNA) and morphology, that is, the virion size, shape, and the presence or absence of an envelope. The host range and immunological properties (serotypes) of the virus are also used. Physical and physicochemical properties such as molecular mass, buoyant density, thermal inactivation, pH stability, and sensitivity to various solvents are used in classification. Virus genera represent groupings of species of viruses that share common characteristics and are distinct from the member viruses of other genera. Virus genera are designated by terms with the suffix-virus. A virus species is defined as a polythetic class of viruses that constitutes a replicating lineage and occupies a particular ecological niche.

Some viral families and their respective, sub-families, genera, and species contemplated for inactivation by contact and adsorption by the clays described herein include, but are not limited to, the following viruses set out in Tables 1-3 below. Reoviridae and its genera rotavirus; poliovirus type 2; enteroviruses; bovine rotavirus; and bovine coronaviruses are excluded from the viruses that are inactivated by the smectite clays described herein.

TABLE 1
DNA VIRUSES

| Family | Sub-Family | Genus | Virus |
|---|---|---|---|
| Herpesviridae | Alphaherpesvirinae | Simplexvirus | Herpes simplex type 1 (HHV-1) |
| | | | Herpes simplex type 2 (HHV-2) |
| | | Varicellovirus | Varicella zoster virus (HHV-3) |
| | Betaherpesvirinae | Cytomegalovirus | Cytomegalovirus virus (HHV-5) |
| | | Roseolovirus | Human herpes virus type 6, 7 |
| | Gammaherpesvirinae | Lymphocryptovirus | Epstein Barr virus (HHV-4) |
| | | Rhadinovirus | Human herpes virus type 8 |

| | | |
|---|---|---|
| Poxviridae | Orthopoxvirus | Variola virus |
| | Molluscipoxvirus | Molluscum contagiousum virus |
| Adenoviridae | Mastadenovirus | Human adenovirus |
| Papovaviridae | Papillomavirus | Papillomavirus |
| | Polyomavirus | BK virus |
| | | JC virus |
| Parvoviridae | Erythrovirus | Human parvovirus (B19) |

TABLE 2
RNA VIRUSES

| Family | Genus | Virus |
|---|---|---|
| Picornaviridae | Rhinovirus | Rhinovirus |
| | Hepatovirus | Hepatitis A virus |
| | Rubivirus | Rubella virus |
| | Alphavirus | Eastern equine encephalitis virus |
| | Rhadinovirus | Human herpes virus type 8 |
| Togaviridae | Flavivirus | Yellow fever virus |
| | | Dengue virus |
| | | West Nile virus |
| Flaviviridae | Hepacvirus | Hepatitis C virus |
| | Coronavirus | Human coronavirus |
| | Calicivirus | Norwalk virus |
| | Rubulavirus | Mumps virus |
| Coronaviridae | Morbillivirus | Measles virus |
| Caliciviridae | Pneumovirus | Respiratory syncitial virus (RSV) |
| Paramyxoviridae | Paramyxovirus | Human parainfluenza virus 1 |
| | Lyssavirus | Rabies virus |
| | Filovirus | Ebola virus |
| | Arenavirus | Lassa fever virus |
| Rhabdoviridae | Influenzavirus A | Influenza A |
| Filoviridae | Influenzavirus B | Influenza B |
| Arenaviridae | Influenzavirus C | Influenza C |
| Orthomyxoviridae | Hantavirus | Sin Nombre virus |
| Bunyaviridae | | |

TABLE 3
DNA-RNA REVERSE TRANSCRIBING VIRUSES

| Family | Genus | Virus |
|---|---|---|
| Retroviridae | Lentivirus | Human immunodeficiency |
| | | viruses |
| | BLV-HTLV | Human T-cell leukemia |
| | retroviruses | viruses |
| Hepadnaviridae | Orthohepadnavirus | Hepatitis B virus |

### Examples

### Example 4

### Antiviral Activity of Test Compounds Against HIV-1

In this study, three different compositions of bentonite clay were studied (R-0088, R-0089, and R-0090) to evaluate their adsorption and antiviral efficacy against an HIV-1 virus (Retroscreen Virology Ltd). Each bentonite clay composition was studied at three different concentrations (0.01% w/v, 0.001% w/v, and 0.0001% w/v) prepared in sterile double-distilled water) and at three different incubation times (1 minute, 5 minutes, and 10 minutes). Test compositions composed of various mineral clays and controls (as listed below) were prepared.
R-0088—purified homoionic sodium bentonite mixture, purified in accordance with U.S. Pat. No. 6,050,509.
R-0089—purified acid activated clay mixture.
R-0091—purified bentonite:dextran analog modified mixture.
C8166 growth media (negative-control)
20% Ethanol/PBS (positive control)

HIV-1IIIB (AL307 with a titer of 104TCID5O/ml) was supplied from the Retroscreen Virology Ltd virus repository. Virucidal and P24 assays were carried out as set out below to evaluate antiviral activity. The p24 antigen assay measures the viral capsid (core) p24 protein in blood that is detectable earlier than HIV antibody during acute infection.

Virucidal Assay

1. 40 μl of the viral stock solution was added to each concentration of test compound (360 μl) and left to incubate at room temperature for the incubation times specified above.
2. The reaction was terminated by the addition of cell infection media (3.6 ml), which diluted the reaction 10-fold.
   P24 Assay
1. The samples were left to settle for 1.5 hours before being added to the P24 antigen coated plates.
2. 200 μl of each sample was added to the assay plate.
3. 110 μl of neat stock virus (AL307) was added to the relevant wells on the plate.
4. Empigen (final concentration of 0.8%) was added to all these wells.
5. The neat stock virus was titrated across the wells following a 10-fold dilution series in RPMI-1640 containing 1% Empigen.
6. The P24 assay was then conducted as instructed in the current Retroscreen Virology Ltd. SOP.

Only R-0088 at 0.01% w/v concentration reduced the viral titer of HIV-1_{IIIB} at the 10 minute incubation time with 99.13% efficacy exhibited. Virucidal results for R-0088 demonstrated that a time-response is exhibited by the 0.01% w/v concentration. At this concentration, the reduction in the HIV1_{IIIB} virus titer was significant at the 10 minute incubation time with a reduction of 2.29 logs. A reduction of ≧1−log₁₀ TCID₅₀/ml (Oxford et al, Antiv. Chem. Chemother. 5:176-181, 1994) is deemed significant for the virucidal assays used in this study, and is equivalent to ≧90% reduction in viral titer. Virucidal results for R-0089 and R-0091 did not demonstrate significant reductions in HIV-1IIIB titer.

At the highest test concentration (0.01% w/v), R-0088 exhibited a significant reduction in the HIV-1_{IIIB} (AL307 with a titer of 10⁴TCID₅₀/ml). R-0089 and R-0091 did not exhibit significant reductions in the HIV1_{IIIB} virus titer for any of the variables tested.

### Example 5

### Antiviral Activity of Test Compounds Against Influenza A

This study was performed to determine whether the test compounds have virucidal efficacy against an epidemic strain of Influenza A virus and to assess the cytotoxic potential of the test compounds on Madin-Darby canine kidney cells (MDCK) cells. Three different compositions of bentonite clay (R-0088, R-0089, and R-0090) were studied to evaluate their adsorption and antiviral efficacy against an Influenza A/Panama/2007/99 (H3N2) virus.

Test compositions composed of various mineral clays and controls (as listed below) were prepared.
R-0088—purified sodium bentonite mixture, purified in accordance with U.S. Pat. No. 6,050,509.
R-0089—purified acid activated clay mixture.
R-0090—purified bentonite-sialic acid mixture.
C8166 growth media (negative control)
20% Ethanol/PBS (positive control)

Each bentonite clay mixture was studied at three different concentrations (0.01% w/v, 0.001% w/v, and 0.0001% w/v prepared in sterile double-distilled water) and at five different incubation times (30 seconds, 1 minute, 5 minutes, 10 minutes, and 30 minutes).

The cells of the toxicity controls were incubated with cell maintenance media, whereas the cells of the virucidal controls were incubated with cell infection media. The stock titer of Influenza A/Panama/2007/99 virus was 7.7 log₁₀ TCID₅₀/ml. Before use in the virucidal assay, the stock virus was diluted 100-fold in infection media. It was then diluted a further 2-fold when it was added to the reaction mixture (section 9.3.2, step 4). The resulting test titer was therefore 5.4 log₁₀ TCID₅₀/ml. The protocols for the toxicity assay and the virucidal assay are set out below.

Toxicity Assay

1. Cells (100 μl/well) at 1×10⁵ cells/ml were seeded onto 96-well plates and incubated at 37° C. for ˜24 hours.
2. The cell maintenance media on the plates was removed and the cell monolayer washed twice with PBS (100 μl/well).
3. Each test compound (100 μl/well) was added, in quadruplicate, to the plate and left to incubate at room temperature for the various times specified.
4. The test compounds were removed, and the cell monolayer washed twice with phosphate buffered saline (PBS) (100 μl/well).
5. Cell-maintenance media (10 μl/well) was added to the cell monolayer and the plates incubated at 37° C. for ˜24 hours
6. A crystal violet assay was performed on the plates in accordance to the Retroscreen Virology Ltd. SOP VA024-01.

Controls utilized in the toxicity assay were:
Cell only control: untreated cells. This was a negative control for toxic cytopathic effect (tCPE) and was also an indicator of cell quality.
Diluent control: cells treated with sterile double-distilled water for the specified times. This was a negative control for the test compounds and assessed any toxic effects of the diluent.
Cell and PBS control: untreated cells washed four times with PBS and incubated with cell maintenance media. This was a negative control for the washing steps, which involved a total of four washes with PBS.
   Virucidal Assay
1. Cells (100 μl/well) at 1×10⁵ cells/ml were seeded onto 96-well plates and incubated at 37° C. for ˜24 hours.
2. The cell maintenance media on the plates was removed and the cell monolayer washed twice with PBS (100 μl/well).
3. Cell infection media (10.0 μl/well) was added to the plates.
4. Diluted virus (20 μl) of 1/2000 viral stock solution was added to each test compound (20 μl) and left to incubate at room temperature for the various incubation times specified.
5. The reaction was terminated by the addition of cell infection media (3.6 ml), which diluted the reaction 10-fold.
6. The termination mixture was centrifuged (4000 rpm for 10 minutes) and the supernatant harvested.
7. The cell infection media in wells B4-B11 of the 96-well plate was removed. The supernatant (111 μl/well) was added to wells B8-B11, and the cell only control was added to wells B4-B7. Both were plated in quadruplicate.
8. The plates were incubated at 37° C. and 5% CO₂ for 2 days.
9. On day 2 post-infection, the plates were scored for viral cytopathic effect (vCPE) and a hemagglutination (HA) assay was performed as per Retroscreen Virology Ltd. SOP VA018-02.

Controls utilized in the virucidal assay were:
Cell only control: cells not infected with virus. This is a negative control for vCPE and is also an indicator of cell quality.
Virus only control: cells infected with a 1/2000 dilution of the virus stock. This was a positive control for vCPE.
Diluent control: cells infected with virus that was pre-treated with sterile double-distilled water for the specified times. This was a negative control for the test compounds and assessed any antiviral effects of the diluent.
Spun virus control: cells infected with virus that was centrifuged at 4000 rpm for 10 minutes. This was a negative control for the centrifugation step and assessed whether centrifugation affected viral titer.
Antiviral control: cells infected with virus pre-treated with citrate buffer at pH3.5. This was a positive control for the test compounds.
For the virucidal assay only, the test compounds were prepared at double the concentrations than those described above. This is due to the 2-fold dilution they underwent when they were mixed with the virus.

The virucidal results demonstrate that a time-response was exhibited by R-0088 at the 0.01% w/v concentration only. At this concentration, the reductions in the Influenza A/Panama/2007/99 virus titer by R-0088 were only significant for the 10 and 30 minute incubation times. R-0089 and R-0090 did not demonstrate significant reductions in the Influenza A/Panama/2007/99 virus titer.

Thus, at the highest test concentration (0.01% w/v), R-0088 exhibited a significant reduction in the Influenza A/Panama/2007/99 virus titer at the 10 and 30 minute incubation times. R-0089 and R-0090 did not exhibit significant reductions in the Influenza A/Panama/2007/99 virus titer for any of the variables tested.

### Example 6

### Antiviral Activity of Additional Test Compounds Against Influenza A

This study was performed to determine whether additional test compounds have virucidal efficacy against an epidemic strain of Influenza A virus and to assess the cytotoxic potential of these test compounds on Madin-Darby canine kidney cells (MDCK) cells. Three different compositions of bentonite clay were studied (R-100, R-101, and R-102) to evaluate their adsorption and antiviral efficacy against an Influenza A/Panama/2007/99 (H3N2) virus.

Test compositions composed of various mineral clays (as listed below) were prepared.
R-100—Crude sodium bentonite clay.
R-101—Sodium bentonite clay having non-smectite impurities removed (as in U.S. Pat. No. 6,050,509, but without the ion exchange steps).
R-102—Purified sodium bentonite clay, purified in accordance with U.S. Pat. No. 6,050,509.
C8166 growth media (negative control)
20% Ethanol/PBS (positive control)

Each bentonite clay mixture was studied at three different concentrations (0.01% w/v, 0.001% w/v, and 0.0001% w/v prepared in sterile double-distilled water) and at three different incubation times (10 minutes, 30 minutes, and 60 minutes).

The cells of the toxicity controls were incubated with cell maintenance media, whereas the cells of the virucidal controls were incubated with cell infection media. The stock titer of Influenza A/Panama/2007/99 virus was 7.4 log₁₀ TCED₅₀/ml. Before use in the virucidal assay, the stock virus was diluted 2000-fold in infection media. It was then diluted a further 2-fold when it was mixed with the test compounds, a further 10-fold when it was mixed with the anti-viral control. The protocols for the toxicity assay and the virucidal assay are set out below.

Toxicity Assay

The toxicity assay was performed as set out in Example 2 except for one modification; in step (1) of the assay, cells were seeded at (100 μl/well) at 5×104 cells/ml.

Controls utilized in the toxicity assay were:
Cell only control: untreated cells. This was a negative control for toxic cytopathic effect (tCPE) and was also an indicator of cell quality.
Diluent control: cells treated with sterile double-distilled water for the specified times. This was a negative control for the test compounds and assessed any toxic effects of the diluent.
PBS wash control: untreated cells washed four times with PBS and incubated with cell maintenance media. This was a negative control for the washing steps, which involved a total of four washes with PBS.
   Virucidal Assay
1. Cells (100 μl/well) at 5×10⁴ cells/ml or 7×10⁴ cells/ml were seeded onto 96-well plates and incubated at 37° C. for ˜24 hours.
2. The cell maintenance media on the plates was removed and the cell monolayer washed twice with PBS (100 μl/well).
3. Cell infection media (100 μl/well) was added to the plates.
4. Diluted virus (200 μl) of 1/2000 viral stock solution was added to each test compound (200 μl) and left to incubate at room temperature for the various times specified. (For the antiviral control, 40 μl of the diluted virus was added to 36 μl of citrate buffer.)
5. The reaction was terminated by the addition of cell infection media (3.6 ml), which diluted the reaction 10-fold.
6. The termination mixture was centrifuged (4000 rpm for 10 minutes) and the supernatant harvested.
7. The cell infection media in wells B4-B11 of the 96-well plate was removed. The supernatant (111 μl/well) was added to wells B8-B11, and the virus only control (1/2000 viral stock solution) was added to wells B4-B7. Both were plated in quadruplicate.
8. The plates were incubated at 37° C. and 5% CO₂ for 2-3 days.
9. On day 2 or 3 post-infection, the plates were scored for vCPE and an HA assay was performed as per Retroscreen Virology Ltd. SOP VA018-02.

Controls utilized in the virucidal assay were:
Cell only control: cells not infected with virus. This is a negative control for vCPE and is also an indicator of cell quality.
Virus only control: cells infected with a 1/2000 dilution of the virus stock. This was a positive control for vCPE.
Diluent control: cells infected with virus that was pre-treated with sterile double-distilled water for the specified times. This was a negative control for the test compounds and assessed any antiviral effects of the diluent.
Antiviral control: cells infected with virus pre-treated with citrate buffer at pH3.5. This was a positive control for the test compounds.

For the virucidal assay only, the test compounds were prepared at double the concentrations than those described above. This is due to the 2-fold dilution they underwent when they were mixed with the virus.

R-100, R-101, and R-102 all exhibited time-dependent response toxicity against MDCK cells. R-100, R-101, and R-102 all exhibited a dose-response activity against Influenza A/Panama/2007/99. All the test concentrations of each test compound exhibited time-dependent response activity against Influenza A/Panama/2007/99. Only the highest test concentration (0.01% w/v) of each test compound exhibited significant reductions in virus titer at every incubation time tested.

The toxicity data generated shows that a time-response, and not a dose-response, was exhibited by the test compounds. This confirms earlier research that the incubation time rather than the test compound concentration is the determining factor of toxicity. It was also observed that the survivability of MDCK cells was also affected by the diluent control, as the values generated for the diluent control and the test compounds were similar.

After examining all the data examining toxicity, viral reduction, and therapeutic index, it was determined that there was a difference between the test compounds, but this difference was only marked when at a concentration of 0.01% w/v. As there was a difference between the toxicity of the test compounds, this suggested that the diluent, which remained consistent between the test compounds, has minimal toxicity. Toxicity and reductions in viral titer increased between R-100, R-101, and R-102 respectively. However small changes in percent toxicity for the 0.01% w/v concentration for all the test compounds had considerable impacts on the therapeutic index values.

In summary, R-102 at the highest concentration (0.01% w/v) affected the greatest reduction in viral titer with the highest therapeutic index.

Virisorb Applications and Examples

| | Example | Method of producing | Examples |
|---|---|---|---|
| 7 | Tissue & Towels | A gel comprised of water, the virucidal | The virucidal agent was a protonated |
| | | agent, and other ingredients known to the | montmorillonite added to deionized water |
| | | art is applied to the substrate that can be | in a concentration of 1% by weight. |
| | | composed of synthetic or natural fibers by | Between 0.0001% and 5% of the virucidal |
| | | either spraying, roll coating, dipping into a | agent, preferably 3% to 5%, is |
| | | trough containing the above described gel. | contemplated although higher |
| | | The final composition would contain the | percentages are useful. The slurry was |
| | | virucidal agent dispersed throughout. | uniformly sprayed onto a disposable |
| | | | “Bounty” towel in an amount equal to 5 |
| | | | times the weight of the original towel. The |
| | | | saturated towel was dried at 60° C. for 1 |
| | | | hour at which time it was determined that |
| | | | the water has been removed and the |
| | | | virucidal agent (protonated |
| | | | montmorillonite) remains on the towel. |
| | | | Other components that could be added to |
| | | | the gel include antimicrobials and |
| | | | disinfectants. |
| 8 | Masks and | The article of the above example is dried | The virucidal agent was a copper |
| | Disposable | by any number of methods well known to | exchanged montmorillonite added to |
| | Medical gowns. | the art. After drying the resultant fabric | deionized water in a concentration of 1% |
| | Air filters, | can be combined with another nonwoven | by weight. Between 0.0001% and 5%, |
| | | material using common laminating | preferably 3% to 5% of the virucidal agent |
| | | techniques. The outer layer of such a | is contemplated although higher |
| | | composition would contain the virucidal | percentages are useful. The slurry was |
| | | composition and can be further converted | uniformly sprayed onto a disposable 3M |
| | | into a disposal mask, air filter, medical | dust mask in an amount equal to 10 times |
| | | gown, bandage, bed pad, arid various | the weight of the original mask. The |
| | | articles of clothing. | saturated mask was dried at 80° C. for 1 |
| | | | hour at which time it was determined-that |
| | | | the water has been removed and the |
| | | | virucidal agent (copper montmorillonite) |
| | | | remained on the towel. Other |
| | | | components that could be added to the |
| | | | gel include antimicrobials, and |
| | | | disinfectants. |
| 9 | Wall paper | The article of the above composition is | |
| | | dried by any number of methods. The | |
| | | composition is combined with another | |
| | | fabric or paper through commonly known | |
| | | laminating methods. The second material | |
| | | containing, on one of its sides, an | |
| | | adhesive that can be activated by any | |
| | | number of solvents. Said composition can | |
| | | then be used in clean room environments | |
| | | as a virus resistant wall covering. | |
| 10 | Wet Wipes | A gel comprised of water, the virucidal | The virucidal agent was a silver |
| | | agent, and other ingredients useful for | exchanged montmorillonite added to |
| | | cleaning surfaces is applied to a substrate | deionized water in a concentration of 1% |
| | | composed of either synthetic or natural | by weight. Between 0.0001% and 5%, |
| | | fibers by either spraying, coating by roller | preferably 1% to 5% of the virucidal agent, |
| | | or slot die, dipping into a trough containing | is contemplated, although higher |
| | | the gel, gravure or flexographic printing, | percentages are useful. The slurry was |
| | | inkjet printing, and other means known to | 1% clay uniformly sprayed onto a |
| | | the art. Said composition is further | nonwoven substrate in an amount equal to |
| | | converted by cutting and folding into a wet | 20 times the weight of the original |
| | | wipe. The wet wipe can then be used to | nonwoven substrate. Other components |
| | | clean various surfaces depositing the gel | that could be added to the gel include |
| | | from the substrate to the surface, | antimicrobials, and disinfectants. |
| | | including human skin, animal skin, wood, | |
| | | metal, and plastic surfaces in hospitals, | |
| | | homes, and office buildings, schools, and | |
| | | similar institutions. Wet wipes could also | |
| | | be used to clean and sanitize medical | |
| | | instruments, such as surgical tools, bed | |
| | | pans, and trays. All surfaces treated with | |
| | | the wet wipe would have the virucidal | |
| | | properties of the virucidal agent. | |
| 11 | Paints for clean | A liquid composition comprised of water, | The virucidal agent in an amount of at |
| | rooms | the virucidal agent and other ingredients | least 0.01% by weight, e.g., 0.01 to 10%, |
| | | known to be useful in paint and coating | is added to a formula containing 10-40% |
| | | applications including but not limited to | pigments, 30-55% water, one or more |
| | | pigments, surfactants, emulsifiers, | latex compounds, such as, vinyl-acetate, |
| | | solvents such as binders composed of, | vinyl-acrylate, acrylate, vinyl-acrylate- |
| | | vinyl acetate, vinyl acrylate, acrylate, | ethylene, and vinyl-ethylene, urethane- |
| | | urethane or combinations thereof; | acrylate emulsions in the amount ranging |
| | | epoxies, polyesters, and other setting | from 5-25%. The above composition |
| | | compounds as well as solvents useful for | can be applied to walls, floors, and other |
| | | enabling their compounding, are applied | surfaces. |
| | | to walls, floors, counter-tops with a roller, | |
| | | brush, or by air or airless spraying | |
| | | methods. The composition upon | |
| | | application will inactivate any viruses on | |
| | | the surfaces it has been applied to. | |
| | | Further after application, the composition | |
| | | will retain the ability to further inactivate | |
| | | any viruses that come in contact with the | |
| | | surfaces in the future. | |
| 12 | Laundry additives | The virucidal agent is combined with | |
| | | zeolites, surfactants, and other ingredients | |
| | | commonly used in a laundry detergent. | |
| | | The composition can then be used as a | |
| | | virucidal agent for cleaning washable | |
| | | materials. | |
| 13 | Absorbent mat | A plurality of fibers are combined with the | |
| | with antimicrobial | virucidal agent and alternatively absorbent | |
| | and virucidal | polymers, antimicrobials and anti- | |
| | capability | bacterials. Additional agents to reduce | |
| | | odor may also be included. The final mat | |
| | | is then capable of absorbing fluids and | |
| | | rendering them non infectious | |
| | | alternatively, the mat can be placed over | |
| | | spills of infectious materials and used to | |
| | | absorb these fluids and render them | |
| | | noninfectious. | |
| 14 | Carpet cleaners | The virucidal agent is combined with talc, | The sodium montmorillonite virucidal |
| | and upholstery | sodium bicarbonate, surfactants, | agent was combined in a weight amount |
| | | fragrances and other ingredients | of 70% with 15% talc and 15% sodium |
| | | commonly used in powdered carpet and | bicarbonate. The mixture was a light |
| | | upholstery cleaners. The composition can | colored free flowing powder and can be |
| | | then be used as a virucidal agent by | sprinkled on carpet or upholstery where it |
| | | pouring or sprinkling on the carpet and | will interact with any virus present, easily |
| | | upholstery where it will interact with the | removes the carpet cleaner and bound |
| | | virus and can be subsequently vacuumed up. | virus molecules as determined by removal |
| | | | of the light colored material. |
| 15 | Condom Coating | A gel comprised of water, the virucidal | To a coating solution comprised of |
| | | agent, anti-agent and other ingredients | glycerine, polyethylene glycol or a mixture |
| | | known to the art is applied to the condom | of water, a humectant and a thickener |
| | | prior to packaging. The final composition | such as hydroxylpropyl cellulose is added |
| | | would contain the virucidal agent | the virucidal agent in a concentration of at |
| | | dispersed throughout. In event of condom | least 0.001% up to 30 wt. %. The coating |
| | | failure, the virucidal agent would interact | solution is then placed on the condom to |
| | | with virus released by the male or virus | completely lubricate the surface. The |
| | | already present in the partner to prevent | mixture may also include anti-spermicidal |
| | | infection of either partner. | agents such as Nonoxynol-9. |
| 16 | Vaginal Gel | A gel, crème, or body heat dissolving | The virucidal agent is incorporated in a |
| | | tablet or suppository comprised of water, | water-based formulation that contains |
| | | the virucidal agent, and other ingredients | greater than 0.001% of the |
| | | known to the art is inserted into the vagina | Montmorillonite and includes thickeners |
| | | prior to sexual activity. The final | for the water, such as xanthane gum or |
| | | composition would contain the virucidal | Carbopol along with humectants like |
| | | agent dispersed throughout. The virucidal | glycerine and propylene glycol. |
| | | agent would interact with virus released by | Alternatively, the virucidal agent could be |
| | | the male or virus already present in the | dispersed in a non-aqueous vehicle like |
| | | partner to prevent infection of either | glycerine, propylene glycol or polyethylene |
| | | partner. The product could also be used | glycol. |
| | | in a douche format to cleanse vaginal area | |
| | | after sexual intercourse and deactivate | |
| | | viruses. | |
| 17 | Hand Sanitizer | A hand sanitizer gel comprised of water, | The formula contained from about 40% to |
| | | the virucidal agent, anti-microbial agent | about 70% by weight ethyl alcohol, 30- |
| | | and other ingredients known to the art is | 60% water, glycerin, Carbomer and 1% by |
| | | applied to the hand to improve sanitation. | weight of the sodium montmorillonite |
| | | The final composition would contain the | virucidal agent. The virucidal agent can |
| | | virucidal agent dispersed throughout. | be in an amount of 0.001% to 15% by |
| | | Viricudal agent would inactivate virus | weight. The formula was rubbed on |
| | | present on the hands. | hands to provide for instant sanitization |
| | | | and inactivation of hand-held viruses. |
| 18 | Gastrointestinal | Virucidal agent our compounds are | |
| | Agent | ingested. In gastrointestinal tract, they | |
| | | interact with viruses and prevent infection. | |
| | | When wastes are expelled, viruses are | |
| | | retained on our materials and prevented | |
| | | from causing secondary infections. | |
| 19 | Nasal Lubricant | A solution/spray of the virucidal agent is | A gel comprised of water and the sodium |
| | | placed into nasal passages where it coats | Montmorillonite agent in a weight |
| | | nasal cells. When a virus contacts the | percentage from 0.00001% to 15%, more |
| | | virucidal agent, it is inactivated and | preferably 1-7%, is combined with non- |
| | | prevents infection. | swelling sodium polyacrylate, know by the |
| | | | trade name CARBOPOL ®. Said gel is |
| | | | placed in a squeeze bottle with a nozzle in |
| | | | its top capable of being safely inserted |
| | | | into the nasal cavity. The gel is sprayed |
| | | | into the nasal passages by squeezing the |
| | | | bottle. The above gel may also contain |
| | | | one or more of the following materials - |
| | | | decongestants, such as Phenylephrine |
| | | | Hydrochloride, and other cold relief, |
| | | | menthol, camphor, sodium chloride, |
| | | | thimerosol and other ingredients known in |
| | | | the art. |
| 20 | Dialysis Filter | The virucidal agent is placed in a filter | The virucidal agent is present in the filter |
| | | canister and blood product is pumped | either as a 100% pure media or diluted |
| | | through the filter. The virucidal agent | 10-90% with a porosity aid. The blood |
| | | interacts with the virus present in the | product is circulated through the filter |
| | | blood product influent to reduce and | where the virucidal agent interacts with |
| | | maintain the viral count at an acceptable | the virus present in the blood product to |
| | | level. The acceptable level is dependent, | maintain the viral count below an |
| | | on the virus desired to be removed. | acceptable level. It is envisioned that a |
| | | | protonated silver exchanged |
| | | | montmorillonite would be an effective |
| | | | virucidal product for dialysis filters. |
| 21 | Spill Containment | The virucidal agent is combined with other | The sodium montmorillonite virucidal, |
| | | absorbent and adsorbent materials such | agent was combined in a weight |
| | | as vermiculite, sodium bentonite, oil | percentage of 50% with 30% oil adsorbent |
| | | adsorbents, polyacrylate superabsorbent | and 20% polyacrylate superabsorbent |
| | | polymers, and surfactants. In the event of | polymer. The mixture was a free flowing |
| | | a spill of a virus containing solution in a | powder and can be poured on an organic |
| | | medical associated laboratory, the | or aqueous based spill where it will |
| | | virucidal agent containing spill | interact with any virus present. |
| | | containment mixture is poured on the spill | Vacuuming easily removes the spill |
| | | area and the liquid as well as the virus is | containment agent as determined by |
| | | contained and cleaned up by shovel, or | removal of the brown colored material. |
| | | sweeping. | |

Gel and Stick Compositions

| | Example | Method of producing |
|---|---|---|
| 22 | Vaginal Inserts/STD's | A liquid composition comprised of water, the virucidal agent, and other |
| | | cosmetically and pharmaceutically acceptable ingredients such as |
| | | glycerin, sorbitol, ethyl alcohol, thickeners such as xanthan gum, and the |
| | | like, surfactants, such as lauryl sulfate, and the like. The composition can |
| | | then be used as a gel for applying on male genetalia, vaginal inserts and |
| | | nasal sprays. |
| 23 | Hand sanitizers | The composition of the above example can be combined with ethyl |
| | | alcohol, and/or other antimicrobials such as triclosan, and/or cetyl |
| | | pyridinium chloride and the like. This composition can be used as an |
| | | instant hand sanitizer with enhanced ability to inactivate viruses. |
| 24 | Nasal Gel/spray | The composition of example 22 can be inserted or sprayed into the nasal |
| | | passages |
| 25 | Cold Sore Treatment | The composition of example 22 can be applied to cold sores to aid in |
| | | reducing the duration of cold sores through inactivating the herpes virus. |
| 26 | Alternative Lip and genitalia | An anhydrous gel containing one or more of anhydrous ingredients |
| | protectant | including waxes, synthetic and natural oils, silicones, petrolatum and the |
| | | virucidal agent are mixed together. The compositing is melted and poured |
| | | into a mold, commonly used to form lip coating products. Upon cooling, |
| | | the materials are removed from the molds and can be used as lipsticks, lip |
| | | balms, vaginal inserts, and the like. |
| 27 | Emulsion | Water containing the virucidal agent, and surfactants and lipophilic |
| | | materials such as waxes, synthetic or natural oils, silicones, |
| | | hydrocarbons, and similar materials can be combined by mixing under |
| | | high shear to create an emulsion. This emulsion can be used directly on |
| | | human skin, animal skin and various surfaces as a virucidal agent. |
| | | Alternatively, the composition can be applied to substrates and dried to |
| | | create a filter, bandage and mask. In addition, the emulsion can be |
| | | applied to a substrate that is further converted into a wet wipe that can be |
| | | used to apply the virucidal composition to various surfaces. |
| 28 | Filter device for removing | The virucidal agent is placed in a cartridge that has a porous cover, or a |
| | virus from fluids | plurality of holes, that enables liquid to flow through the cartridge, but |
| | | retain the virucidal agent within it. The device can then be used to |
| | | inactivate viruses in the blood stream of animals or humans, water, and |
| | | any other liquid that may contain viruses. |
| 29 | Blood adsorbent with virus | The virucidal agent is combined with absorbent polymers and other anti |
| | inactivatioin | microbial or antibacterial agents, such as CPC, triclosan, and the like. |
| | | The powder is then capable of solidifying liquid and semi-solid wastes |
| | | from animals and humans and inactivating viruses present in the wastes, |
| | | eliminating the potential for spreading infectious diseases. |

1. A method of inactivating a virus selected from the group consisting of herpesviridae, poxyiridae, adenoviridae, papovaviridae; parvoviridae, picornaviridae, togaviridae, flaviviridae, coronaviridae, caliciviridae, paramyxoviridae, rhabdoviridae, filoviridae, arenaviridae, orthomyxoviridae, bunyaviridae, retroviridae, hepadnaviridae, and combinations thereof comprising contacting the virus with a layered phyllosilicate material for a period of time sufficient to bind at least 90% of the virus molecules onto the layered phyllosilicate material.

2. A method in accordance with claim 1, wherein the virus is selected from the group consisting of simplexvirus, varicellovirus, cytomegalovirus, roseolovirus, lymphocryptovirus, rhadinovirus, orthopoxvirus, molluscipoxvirus, mastadenovirus, papillomavirus, polyomavirus, erythrovirus, rhinovirus, hepatovirus, rubivirus, alphavirus, rhadinovirus, flavivirus, hepacvirus, coronavirus, calicivirus, rubulavirus, morbillivirus, pneumovirus, paramyxovirus, lyssavirus, filovirus, arenavirus, influenzavirus A, influenzavirus B, influenzavirus C, hantavirus, lentivirus, BLV-HTLV retroviruses, orthohepadnavirus, and combinations thereof.

3. A method in accordance with claim 2, wherein the virus is selected from the group consisting of virus herpes simplex type 1 (HHV-1), herpes simplex type 2 (HHV-2), varicella zoster virus (HHV-3), cytomegalovirus virus (HHV-5), human herpes virus type 6, 7, Epstein Barr virus (HHV-4), human herpes virus type 8, variola virus, molluscum contagiousum virus, human adenovirus, papillomavirus, BK virus, JC virus, human parvovirus (B 19), rhinovirus, hepatitis A virus, rubella virus, eastern equine encephalitis virus, human herpes virus type 8, yellow fever virus, dengue virus, west Nile virus, hepatitis C virus, human coronavirus, Norwalk virus, mumps virus, measles virus, respiratory syncitial virus (RSV), human parainfluenza virus 1, rabies virus, ebola virus, lassa fever virus, influenza A, influenza B, influenza C, sin nombre virus, human immunodeficiency viruses, human T-cell leukemia viruses, hepatitis B virus, and combinations thereof.

4. A method in accordance with claim 1, wherein the virus is an Influenza virus.

5. A method in accordance with claim 4, wherein the virus is an Influenza A virus.

6. A method in accordance with claim 1, wherein the virus is an HIV virus.

7. A method in accordance with claim 1, wherein the virus is a combination of an influenza virus and an HIV virus.

8. A method in accordance with claim 7, wherein the Influenza virus is an Influenza A virus.

9. A method in accordance with claim 1, wherein the layered phyllosilicate material is contained in or on a face mask that covers a wearer's nostrils and mouth.

10. A method in accordance with claim 9, wherein the layered phyllosilicate material has at least 90% homoionic interlayer exchangeable cations, in relation to all interlayer exchangeable cations, and has a particle size less than 74 μm.

11. A method in accordance with claim 9, wherein the layered phyllosilicate is sprayed onto the face mask from a suspension: of the layered phyllosilicate in a liquid carrier.

12. A method in accordance with claim 10, wherein the layered phyllosilicate is sprayed onto the face mask from a suspension of the layered phyllosilicate in a liquid carrier.

13. A method of inactivating a virus comprising contacting the virus with a layered phyllosilicate material having at least 90% homoionic interlayer exchangeable cations, in relation to all interlayer exchangeable cations, and having a particle size less than 74 μm, for a period of time sufficient to bind at least 90% of the virus onto the layered phyllosilicate material.

14. A method in accordance with claim 13, wherein the homoionic cations are sodium.

15. A method in accordance with claim 13, wherein the particle size of the phyllosilicate material is less than 50 μm.

16. A method in accordance with claim 15, wherein the particle size of the phyllosilicate material is less than 20 μm.

17. A method in accordance with claim 13, wherein the homoionic interlayer exchangeable cations are protonated onium ions.

18. A method in accordance with claim 13, wherein the virus is an Influenza virus.

19. A method in accordance with claim 18, wherein the virus is an Influenza A virus.

20. A method in accordance with claim 13, wherein the virus is an HIV virus.

21. A method in accordance with claim 13, wherein the phyllosilicate inactivates both an influenza virus and an HIV virus.

22. A method in accordance with claim 21, wherein the influenza virus is an Influenza A virus.

23. A method of inactivating a virus comprising contacting the virus with a layered phyllosilicate material having a particle size wherein at least 99% of the phyllosilicate particles have a particle size less than 20 μm and the virus being inactivated is other than a reovirus, to bind the virus onto the phyllosilicate particles.

24. A method in accordance with claim 23, wherein the phyllosilicate material has interlayer exchangeable cations that are predominantly Na cations.

25. A method in accordance with claim 24, wherein the phyllosilicate material has interlayer exchangeable cations that are predominantly protonated onium ions.

26. A method in accordance with claim 23, wherein the virus is an Influenza virus.

27. A method in accordance with claim 26, wherein the virus is an Influenza A virus.

28. A method in accordance with claim 23, wherein the virus is an HIV virus.

29. A method in accordance with claim 28, wherein the phyllosilicate material inactivates both an influenza virus and an HIV virus.

30. A method of inactivating a virus comprising contacting the virus with exfoliated smectite clay platelets and/or tactoids thereof, to bind the virus onto the smectite clay platelets and/or tactoids.

31. A method in accordance with claim 30, wherein the exfoliated smectite clay comprises predominantly individual smectite clay platelets.

32. A method in accordance with claim 30, wherein the exfoliated smectite clay platelets and/or tactoids are dispersed in a liquid carrier selected from the group consisting of water, an organic solvent, and a combination thereof.

33. A method in accordance with claim 28, wherein the clay platelets and/or tactoids are bound in or bound on a face mask that covers a wearer's nostrils and mouth.

34. A method in accordance with claim 33, wherein the clay platelets and/or tactoids are sprayed onto the face mask from a suspension of the clay platelets and/or tactoids in a liquid carrier.

35. A method of inactivating air-borne viruses in a building by providing a layered phyllosilicate material as a portion of an HVAC building filter media for contact with HVAC-treated air such that the air-borne viruses pass through the layered phyllosilicate material contained in or on the filter media.

36. A method in accordance with claim 35, wherein the phyllosilicate material has at least 90% homoionic interlayer exchangeable cations, in relation to all interlayer exchangeable cations, and has a particle size less than 74 μm.

37. A method in accordance with claim 35, wherein the phyllosilicate material comprises exfoliated phyllosilicate platelets and/or tactoids thereof.

38. A method of inactivating a virus entering nostrils of a person comprising spraying a liquid suspension of a layered phyllosilicate material into the nostrils, thereby coating at least a portion of the person's nasal cells with said phyllosilicate material such that a virus entering the person's nostrils are inactivated by contact with phyllosilicate material.

39. A method in accordance with claim 38, wherein the layered phyllosilicate material has at least 90% homoionic interlayer exchangeable cations, in relation to all interlayer exchangeable cations, and has a particle size less than 74 μm.

40. A method in accordance with claim 38, wherein the phyllosilicate material comprises a liquid suspension of exfoliated-platelets and/or tactoids of the layered phyllosilicate material.

41. A method of removing a virus from a person's blood stream comprising passing the blood through a filter media containing a layered phyllosilicate material.

42. A method in accordance with claim 41, wherein the layered phyllosilicate material has at least 90% homoionic interlayer exchangeable cations, in relation to all interlayer exchangeable cations, and has a particle size less than 74 μm.

43. A method in accordance with claim 41, wherein the layered phyllosilicate material comprises exfoliated platelets and/or tactoids of the layered phyllosilicate material.

44. A method of preventing a sexually transmittable virus from one sexual partner from infecting another sexual partner comprising inserting a layered phyllosilicate material into an intended sexual orifice of one of the sexual partners.

45. A method in accordance with claim 44, wherein the intended sexual orifice is a vagina.

46. A method in accordance with claim 44, wherein the layered phyllosilicate material has at least 90% homoionic interlayer exchangeable cations, in relation to all interlayer exchangeable cations, and has a particle size less than 74 μm.

47. A method in accordance with claim 46, wherein the intended sexual orifice is a vagina.

48. A method in accordance with claim 44, wherein the layered phyllosilicate material comprises exfoliated platelets and/or tactoids of a smectite clay.

49. A method in accordance with claim 48, wherein the intended sexual orifice is a vagina.

50. A method of preventing a sexually transmittable virus from one sexual partner from infecting another sexual partner comprising coating a condom, worn by one of the sexual partners, with a layered phyllosilicate material.

51. A method in accordance with claim 50, wherein the layered phyllosilicate material has at least 90% homoionic interlayer exchangeable cations, in relation to all interlayer exchangeable cations, and has a particle size less than 74 μm.

52. A method in accordance with claim 50, wherein the phyllosilicate material comprises exfoliated platelets and/or tactoids of the layered phyllosilicate material.

53. A method of inactivating a virus in a gastrointestinal tract of a person comprising having the person ingest a layered phyllosilicate material.

54. A method in accordance with claim 53, wherein the layered phyllosilicate material has at least 90% homoionic interlayer exchangeable cations, in relation to all interlayer exchangeable cations, and has a particle size less than 74 μm.

55. A method in accordance with claim 53, wherein the phyllosilicate material comprises exfoliated platelets and/or tactoids of the layered phyllosilicate material.

56. A method of inactivating a virus on a person's hands comprising contacting the person's hands with a layered phyllosilicate material, suspended in a liquid carrier.

57. A method in accordance with claim 56, wherein the layered phyllosilicate material has at least 90% homoionic interlayer exchangeable cations, in relation to all interlayer exchangeable cations, and has a particle size less than 74 μm.

58. A method in accordance with claim 56, wherein the layered phyllosilicate material comprises exfoliated platelets and/or tactoids of the layered phyllosilicate material.

59. A method of inactivating a virus on a surface of a substrate comprising contacting the surface of the substrate with a substrate wiping material containing a layered phyllosilicate material.

60. A method in accordance with claim 59, wherein the layered phyllosilicate material has at least 90% homoionic interlayer exchangeable cations, in relation to all interlayer exchangeable cations, and has a particle size less than 74 μm.

61. A method in accordance with claim 59, wherein the layered phyllosilicate material comprises exfoliated platelets and/or tactoids of the layered phyllosilicate material.
